(19) United States
(12) Patent Application Publication
(10) Pub. No.: US 2025/0200407 A1
(43) Pub. Date: Jun. 19, 2025

(54) Feature Selection Method and System Based on Fuzzy Label Relaxation

(71) Applicant: THE HONG KONG POLYTECHNIC UNIVERSITY, Hong Kong (CN)

(72) Inventors: Jing CAI, Hong Kong (CN); Yuanpeng ZHANG, Hong Kong (CN)

(21) Appl. No.: 18/937,159

(22) Filed: Nov. 5, 2024

(30) Foreign Application Priority Data
Dec. 18, 2023 (CN) 202311743438.4

(51) Int. Cl. G06N 7/02

(52) U.S. Cl. CPC G06N 7/023

(57) ABSTRACT

The present disclosure provides a feature selection method based on fuzzy label relaxation, which comprises the following steps: (a) acquiring sample data, and performing feature extraction on the sample data to obtain a feature data matrix; (b) learning a fuzzy membership to obtain a fuzzy membership matrix; (c) performing soft relaxation on a label matrix with the fuzzy membership matrix, and constraining a feature selection matrix to be row sparse; and (d) based on the steps (b) and (c), obtaining an objective function of the feature selection based on label fuzzy relaxation, and solving the objective function. In addition, the present disclosure further provides a feature selection system based on label fuzzy relaxation.

### CROSS-REFERENCE TO RELATED APPLICATIONS

This application claims priority to China Patent Application No. 202311743438.4 filed on Dec. 18, 2023, the disclosure of which is hereby incorporated by reference in its entirety.

### TECHNICAL FIELD

The present disclosure generally relates to a feature selection method based on fuzzy label relaxation and a feature selection system based on fuzzy label relaxation.

### BACKGROUND

Nowadays, collected data usually has a high-dimensional feature expression, in order to describe a target object more comprehensively. Many machine learning problems become quite difficult when the dimensionality of data is high. The number of possible configurations with a different set of variables will increase exponentially with the increase of the number of variables. Researchers have proposed feature selection methods and techniques in order to avoid the curse of dimensionality and reduce data noise in high-dimensional samples. Feature selection is mainly to perform filtering and sorting on high-dimensional features of samples to obtain important features and improve the accuracy of classifiers.

CN111652271A discloses a nonlinear feature selection method based on a neural network. In the method, the linear error function in sparse regularization is changed into a neural network error function, and group sparsity constraints are applied to the weights of the input layer of a neural network according to the complexity of the weights of the neural network, in order to improve the prediction accuracy of a sparse regularization model for nonlinear problems. In addition, the neural network is solved by using a L_{2,1} norm as an error function to reduce the influence of outliers on the result of feature selection.

CN111931562A discloses an unsupervised feature selection method and system based on soft label regression. In the method, soft labels of data samples are learned based on fuzzy clustering, a feature selection matrix is learned by means of a sparse regression model, thus soft label learning is associated with feature selection matrix learning, a more discriminating feature subset in the sample data is solved, and the accuracy of a prediction model is improved.

CN113869454A discloses a sparse feature selection method for hyperspectral images based on fast embedded spectral analysis. In the method, a F norm regular term is introduced to keep the manifold structure of data and the class information of subspaces as far as possible; and a L_{2,0} norm constraint is introduced to enhance the sparsity constraint of subspaces, which is helpful for obtaining a feature subset having the richest class information.

The above-mentioned patent documents are incorporated herein by reference. Although some patent documents have disclosed feature selection of data using regularization learning and soft label learning, it can be seen from the technical schemes of the inventions that the inventions still have some shortcomings: 1) hard labels are usually used in the process of model learning, resulting in weakened association between classes and neglection of many potential semantics; 2) an unsupervised learning method only analyzes the relationship between features, but can't use the important information carried by sample labels; 3) in the process of classification, a strict binary label matrix or linear model is used for sample points at the decision boundary, but that method is too tough to reflect the characteristics of real-world data.

### SUMMARY

The present invention provides a method for learning a class structure of training samples in a high-dimensional feature space with a fuzzy unsupervised learning method, expressing the class structure by means of a fuzzy membership, performing label relaxation by means of the fuzzy membership, and incorporating a supervised embedded feature selection framework for solution optimization.

According to an aspect of the present invention, the present invention provides a feature selection method based on fuzzy label relaxation, which comprises the steps of:
(a) acquiring sample data, and performing feature extraction on the sample data to obtain a feature data matrix;
(b) learning a fuzzy membership to obtain a fuzzy membership matrix;
(c) performing soft relaxation on a label matrix with the fuzzy membership matrix, and constraining a feature selection matrix to be row sparse; and
(d) based on the steps (b) and (c), obtaining an objective function of the feature selection based on fuzzy label relaxation, and solving the objective function.

In certain embodiments, in the step (b), the fuzzy membership is learned with the following method:

$$\begin{matrix}{min}_{{o}_{j} , H} {\sum }_{i = 1}^{n} {\sum }_{j = 1}^{c} {h}_{ij} {‖ {x}_{i} - {o}_{j} ‖}_{2}^{2} + α {‖ H ‖}_{F}^{2} \\ s . t . {\sum }_{j = 1}^{c} {h}_{ij} = 1 , 0 \leq {h}_{ij} \leq 1\end{matrix}$$

where xᵢ is the ith sample data, oⱼ is the jth class center of the sample data, and hᵢⱼ is a membership between the ith sample and the jth class center; the second term uses a square F norm to apply a regularization constraint on a membership matrix, H∈R^{n×c} is a membership matrix of the sample data features, and a is a regularization parameter of the second term in the above expression.

In certain embodiments, in the step (c), the objective function for the soft relaxation of the label matrix is as follows:

$${min}_{P} {‖ XP - ( Y + H ) ‖}_{F}^{2} + γ {‖ P ‖}_{2 , 1}$$

where the first term is a loss function, X∈R^{n×d} is a feature data matrix, P∈R^{d×c} is a feature selection matrix, Y∈R^{n×c} is a label matrix, and H∈R^{n×c} is the membership matrix of the sample data features; the second term applies a penalty with a L_{2,1} norm on the feature selection matrix, and γ is a regularization parameter of the second term in the above expression.

In certain embodiments, in the step (d), the objective function of the feature selection based on label fuzzy relaxation is as follows:

$$\begin{matrix}J = {min}_{{o}_{j} , H , P} ( {\sum }_{i = 1}^{n} {\sum }_{j = 1}^{c} {h}_{ij} {‖ {x}_{i} - {o}_{j} ‖}_{2}^{2} + α {‖ H ‖}_{F}^{2} ) + λ ( {‖ XP - ( Y + H ) ‖}_{F}^{2} + γ {‖ P ‖}_{2 , 1} ) \\ s . t . {\sum }_{j = 1}^{c} {h}_{ij} = 1 , 0 \leq {h}_{ij} \leq 1\end{matrix}$$

where xᵢ is the ith sample data, oⱼ is the jth class center of the sample data, hᵢⱼ is the membership between the ith sample and the jth class center, and H∈R^{n×c} is the membership matrix of the sample data features; the second term is a loss function and a term for generalization, X∈R^{n×d} is the feature data matrix, P∈R^{d×c}, is the feature selection matrix, Y∈R^{n×c} is the label matrix, and α, γ and λ are regularization parameters.

In certain embodiments, the objective function in the step (d) is solved with an alternative optimization method.

In certain embodiments, in the alternative optimization method, an update rule of P is as follows:

$$L = {arg min}_{P} {‖ XP - ( Y + H ) ‖}_{F}^{2} + γ {‖ P ‖}_{2 ,}$$

The expression is transformed into the following equivalent form:

$${arg min}_{P} {‖ XP - ( Y + H ) ‖}_{F}^{2} + γ tr ( {P}^{T} Γ P ) .$$

By taking a derivative of the above expression with respect to P and setting the derivative to 0, P=(X^{T}X+γΓ)⁻¹X^{T}F is obtained, where F=(Y+H).

In certain embodiments, in the alternative optimization method, an update rule of oⱼ is as follows:

$$L = {arg min}_{{o}_{j}} {\sum }_{i = 1}^{n} {\sum }_{j = 1}^{c} {h}_{ij} {‖ {x}_{i} - {o}_{j} ‖}_{2}^{2} .$$

By taking a derivative of the above expression with respect to oⱼ and setting the derivative to 0,

$${o}_{j} = \frac{{\sum }_{i = 1}^{n} {h}_{ij} {x}_{i}}{{\sum }_{i = 1}^{n} {h}_{ij}}$$

is obtained.

In certain embodiments, in the alternative optimization method, an update rule of H is as follows:

$$\begin{matrix}arg {min}_{H} ( {\sum }_{i = 1}^{n} {\sum }_{j = 1}^{c} {h}_{ij} {‖ {x}_{i} - {o}_{j} ‖}_{2}^{2} + α {‖ H ‖}_{F}^{2} ) + λ {‖ XP - ( Y + H ) ‖}_{F}^{2} \\ s . t . {\sum }_{j = 1}^{c} {h}_{ij} = 1 , 0 \leq {h}_{ij} \leq 1.\end{matrix}$$

The expression is simplified to the following form by setting dᵢⱼ=∥xᵢ−oⱼ∥₂², and R=XP−Y:

$$\begin{matrix}arg {min}_{H} {\sum }_{j = 1}^{c} ( {d}_{ij} {h}_{ij} + {α ( {h}_{ij} )}^{2} ) + λ {‖ R - H ‖}_{F}^{2} \\ s . t . {\sum }_{j = 1}^{c} {h}_{ij} = 1 , 0 \leq {h}_{ij} \leq 1.\end{matrix}$$

Furthermore, the expression is transformed to the following vector form:

$$\begin{matrix}{arg min}_{{h}_{i}} {‖ {h}_{i} - \frac{{d}_{i}}{2 α} ‖}_{2}^{2} + λ {‖ {r}_{i} - {h}_{i} ‖}_{2}^{2} \\ s . t . {\sum }_{j = 1}^{c} {h}_{ij} = 1 , 0 \leq {h}_{ij} \leq 1.\end{matrix}$$

After the expression is sorted, a Lagrange function of the expression is as follows:

$$L ( {h}_{i} , η , {θ}_{i} ) = \frac{1}{2} {‖ {h}_{i} + \frac{{d}_{i}}{2 α} ‖}_{2}^{2} + \frac{λ}{2} {‖ {r}_{i} - {h}_{i} ‖}_{2}^{2} - η ( {\sum }_{j = 1}^{c} {h}_{ij} - 1 ) - {θ}_{i}^{T} {h}_{i}$$

where both η and θ are Lagrange coefficients, and a solution of H can be obtained in the following way according to Karush-Kuhn-Tucker conditions:

$${h}_{i} = {( \frac{λ {r}_{i} - \frac{{d}_{i}}{2 α} + η}{1 + λ} )}_{+}$$

where the function ( )⁺ indicates (a)⁺=max(0, a).

In certain embodiments, the objective function in the step (d) is solved with the following pseudo codes:
inputs: X∈R^{n×d}, Y∈R^{n×c}, and regularization parameters α, γ and λ;
outputs: a feature selection matrix P∈R^{d×c} and nSel features;
repeat the following steps (1)-(4):
step (1): update the fuzzy class center oⱼ;
step (2): update the membership matrix H;
step (3): update the feature selection matrix P, until (∥obj(t)−obj(t−1)∥≤10⁻⁵).
step (4): output the feature selection matrix P,
calculate ∥pᵢ∥₂, (i=1, 2, . . . , d) according to the feature selection matrix P, and then carry out sorting in a descending order, and take the first nSel features as selected features.

According to another aspect of the present invention, the present invention provides a feature selection system based on fuzzy label relaxation, which comprises:
a data set acquisition module configured to acquire sample data and perform feature extraction on the sample data to obtain a feature data matrix;
a fuzzy membership learning module configured to learn a fuzzy membership to obtain a fuzzy membership matrix;
a label matrix soft relaxation module configured to perform soft relaxation on a label matrix with the fuzzy membership matrix and constrain the feature selection matrix to be row sparse; and
an objective function solving module, which, based on the fuzzy membership learning module and the label matrix soft relaxation module, obtains an objective function of the feature selection based on fuzzy label relaxation, and solves the objective function.

In certain embodiments, the system utilizes the method mentioned above in the first aspect and embodiments.

The Summary is provided to introduce the selection of concepts in a simplified form, and the concepts will be further described in the following Detailed Description. The Summary is not intended to identify key features or essential features of the claimed subject matter or to be used as an aid in defining the scope of the claimed subject matter. Other aspects and advantages of the present invention will be described in the Detailed Description.

### BRIEF DESCRIPTION OF THE DRAWINGS

The accompanying drawings include drawings plotted to further illustrate and elaborate the above and other aspects, advantages and features of the present disclosure. It should be appreciated that these drawings only depict some embodiments of the present disclosure, but are not intended to limit the scope of the present disclosure. Now, the present disclosure will be described and explained with additional particularities and details with reference to the accompanying drawings, in which:

FIG. 1 shows a flowchart of a supervised feature selection method based on fuzzy label relaxation according to embodiments of the present invention;

FIG. 2 shows the result of an ablation experiment according to embodiments of the present invention; and

FIG. 3 shows the result of generalization performance according to embodiments of the present invention.

### DETAILED DESCRIPTION

Based on fuzzy theory and label relaxation technology, the present disclosure provides a feature selection method based on fuzzy label relaxation and a feature selection system based on fuzzy label relaxation.

Benefits, advantages, solutions to problems, and any element that may give rise to any benefit, advantage or solution or make any benefit, advantage, or solution more obvious should not be construed as key, indispensable or essential features or elements of any or all claims. The present invention is only defined by the appended claims, which may include any modification made during the pendency of the present application and all equivalents of those claims.

In the following claims and the previous description of the present invention, unless the context specifies otherwise owing to the expressive language or necessary meaning, the term “comprise” or any variant thereof, such as “comprising”, is used in an inclusive sense, i.e. to specify the existence of the stated feature, but does not exclude the existence or addition of other features in various embodiments of the present invention.

Feature selection refers to a process of selecting a subset of related features (i.e. attributes and metrics) in order to build up a model. The objective of feature selection is to find an optimal feature subset. Feature selection can weed out unrelated or redundant features, thereby reduce the number of features, improve the accuracy of the model, reduce the running time, and improve the generalization ability of the model. By selecting different evaluation metrics, feature selection algorithms can be categorized into three categories, namely, wrapper algorithm, filter algorithm and embedded algorithm. A wrapper algorithm scores a feature subset, and calculates the number of errors (the error rate of the model) on a reserved set with the model trained with the subset to get a score of the subset. A filter algorithm uses proxy metrics instead of the error rate to score a feature subset. An embedded algorithm performs feature selection in the model building process, in which the learning algorithm uses its own variable selection process, and feature selection and algorithm training are carried out simultaneously. For example, the machine learning algorithm and model are used to train to obtain weight coefficients of the features, the weight coefficients are sorted in a descending order, and features having greater weight coefficients are selected, wherein the weight coefficients often represent contributions or significances of the features to the model.

A core problem in machine learning is to design an algorithm that not only performs well on training data, but also is generalized well on new inputs. Many strategies are explicitly designed to reduce test errors (possibly at the expense of increased training errors). Those strategies are collectively referred to as regularization. Regularization is one of the tools commonly used to decrease the risk of over-fitting. Regularization may be defined as “a modification to the learning algorithm for the purpose of reducing generalization errors rather than training errors”. For example, additional constraints may be added to the machine learning model to constrain the parameter values, or additional terms may be added to the objective function to apply soft constraints on the parameter values.

Many regularization methods constrain the learning ability of a model (e.g., neural network, linear regression, or logistic regression) by adding a parameter norm penalty Ω(θ) to the objective function J. The regularized objective function is denoted as \tilde{J}:

$$\tilde{J} ( θ ; X ; y ) = J ( θ ; X ; y ) + α Ω ( θ )$$

where X is a m×n design matrix, y is an associated target, and θ contains all parameters (weights and offsets). Weights may be comprehended as the degrees of influences of individual features on the prediction. α∈[0, ∞) is a hyper-parameter that weights the relative contributions of the norm penalty term Ω and the standard objective function J(X; θ). If α is set to 0, it means that there is no regularization. The greater the value of a is, the greater the corresponding regularization penalty will be. When the regularized objective function \tilde{J} is minimized with our training algorithm, the error of the original objective function J on the training data will be reduced, and the scale of the parameter set θ (or a parameter subset) under some metrics will be reduced as well. When a different parameter norm Ω is selected, a different solution will be favored. A norm may be comprehended as a function that maps an object to a non-negative real number.

Among the norms of a vector x, the L₀ norm refers to the number of non-zero elements in the vector. For example, if x=[0, 1, 1, 0, 0, 1], then ∥x∥₀=3. The L_{P} norm is defined as follows, where p∈R, p≥1:

$${‖ x ‖}_{p} = {( {\sum }_{i} {\vert {x}_{i} \vert}^{p} )}^{\frac{1}{p}} .$$

Intuitively, the norms of a vector x measure the distance from the origin to a point x. When p=1, the L₁ norm is the sum of the absolute values of the elements in the vector:

$${‖ x ‖}_{1} = {\sum }_{i} \vert {x}_{i} \vert .$$

Both the L₀ norm and the L₁ norm can describe the sparsity of the vector. Sparsity means that some parameters in the optimal value are 0. When p=2, the L₂ norm is referred to as a Euclidean norm:

$${‖ x ‖}_{2} = \sqrt{{\sum }_{i} {{x}_{i}}^{2}} .$$

The L₂ norm may be simplified as ∥x∥, i.e. the subscript 2 is omitted. In addition, a square L₂ norm is also often used to measure the magnitude of a vector, and is calculated as a dot product x^{T}x and denoted as ∥x∥₂²:

$${‖ x ‖}_{2}^{2} = {\sum }_{i} {{x}_{i}}^{2} .$$

A L₂ parameter norm penalty can reduce the weights of the features that have a smaller covariance with the outputted objective by adding a regular term to the objective function. To measure the magnitude of a A∈R^{m×n}, the following F norm may be used, where aᵢⱼ is the element in row i and column j of the matrix A:

$${‖ A ‖}_{F} = \sqrt{{\sum }_{i = 1}^{m} {\sum }_{j = 1}^{n} {a}_{i , j}^{2} .}$$

In addition, the square F norm is defined as follows:

$${‖ A ‖}_{F}^{2} = {\sum }_{i = 1}^{m} {\sum }_{j = 1}^{n} {a}_{i , j}^{2} .$$

For a matrix A∈R^{m×n}, the L_{2,1} norm is defined as follows:

$${‖ A ‖}_{2 , 1} = {\sum }_{i = 1}^{m} \sqrt{{\sum }_{j = 1}^{n} {a}_{i , j}^{2}} = {\sum }_{i = 1}^{m} {‖ {a}_{i , :} ‖}_{2} .$$

The inverse (or inverse number) of a matrix B∈R^{n×n} refers to the sum of the elements in the principal diagonal, and may be expressed as:

$$tr ( B ) = {\sum }_{i} {b}_{i , i} .$$

Thus, for a matrix A∈R^{m×n},

$${‖ A ‖}_{F} = \sqrt{tr ( {AA}^{T} )} = \sqrt{tr ( {A}^{T} A )}$$

and ∥A∥_{2,1}=tr(A^{T}ΓA) are true, where Γ is a diagonal matrix and the iₜₕ diagonal element is

$$\frac{1}{\sqrt{{A}_{i :} {A}_{i :}^{T}} + ε} ,$$

where ε is a constant small enough to avoid a situation that ∥A_{i:1}∥₂ is 0. Aᵢ: is a row vector of the matrix A. The inverse of a matrix has the following characteristics: tr(A+C)=tr(A)+tr(C) and tr(AC)=tr(C)tr(A).

In addition, machine learning algorithms may be generally categorized into unsupervised algorithms and supervised algorithms. Unsupervised learning uses unlabeled data for the training, to learn structural characteristics useful on the data set. Unsupervised learning generates an entire probability distribution of a data set, such as density estimation, synthesis or denoising, or clustering. A supervised learning algorithm learns how to associate inputs with outputs based on a given training set of inputs and outputs, wherein the samples must be annotated and labeled (tagged) artificially. The labels may be certain inputted attributes or targets. A common method for representing a data set is a design matrix X∈R^{n×d}. Each row of the design matrix X contains a different sample. Each column of the design matrix X corresponds to a different feature. The selected features correspond to a feature selection matrix P∈R^{d×c}. In supervised learning, a sample contains a label or target and a set of features. Usually, a label matrix Y∈R^{n×c} may be designed when dealing with a data set that contains a design matrix X of observed features.

Some concepts that are helpful for understanding the present invention have been introduced above. In the real world, a lot of information is ambiguous, and there is no clear judging basis for many things. In order to describe the uncertain relationship between target objects more accurately, it is necessary to find a label relaxation matrix that is more suitable for the realistic scenario and can flexibly represent the probability of membership. Based on an assumption that n samples in a data set contain c potential semantic classes, a label relaxation matrix of the samples may be learned by calculating the degree of membership of each sample to each potential semantic class. Theoretically, the shorter the distance of a sample from the learned class center, the greater the assigned degree of membership should be, i.e. the higher the similarity will be. In the present invention, a Euclidean distance is used to measure the correlation between a sample and a class center. The expression is as follows:

$${min}_{{o}_{j} , H} {\sum }_{i = 1}^{n} {\sum }_{j = 1}^{c} {h}_{ij} {‖ {x}_{i} - {o}_{j} ‖}_{2}^{2} + α {‖ H ‖}_{F}^{2} \\ s . t . \\ {\sum }_{j = 1}^{c} {h}_{ij} = 1 , 0 \leq {h}_{ij} \leq 1 \qquad ( 1 )$$

where xᵢ represents the ith sample data, oⱼ represents the jth class center of the sample data, and hᵢⱼ represents a membership between the ith sample and the jth class center; the second term applies a regularization constraint on a membership matrix by using a square F norm, H∈R^{n×c} is a membership matrix of the sample data features, and α is a regularization parameter of this term.

In the process of pattern recognition, a strict binary label matrix or linear model is used for the sample points at the decision boundary (the boundary used to divide classes in statistical classification). Such a handling method is too tough to reflect the characteristics of real-world data well. Based on the fuzzy relaxation technology, the present invention utilizes a fuzzy membership matrix to perform soft relaxation on a label matrix. In the context of potential semantic information, the association between the classes can be further utilized to improve the accuracy of the model. The expression for soft relaxation of a label matrix is as follows:

$${min}_{P} {‖ XP - ( Y + H ) ‖}_{F}^{2} + γ {‖ P ‖}_{2 , 1} \qquad ( 2 )$$

where

$${min}_{P}$$

represents a minimum value of the following expression obtained by adjusting the independent variable p. The first term is a loss function; X∈R^{n×d} is a feature data set, and feature extraction is performed on each acquired sample data xᵢ to obtain a feature data matrix; P∈R^{d×c} is a feature selection matrix; Y∈R^{n×c} is a label matrix; H∈R^{n×c} is a membership matrix of sample data features. The second term applies a L_{2,1} norm penalty on the feature selection matrix P, so that the feature selection matrix P is constrained to be row sparse; and γ is a regularization parameter of this term.

Based on the expressions (1) and (2), the objective function of feature selection based on fuzzy label relaxation provided by the invention is as follows:

$$J = {min}_{{o}_{j} , H , P} ( {\sum }_{i = 1}^{n} {\sum }_{j = 1}^{c} {h}_{ij} {‖ {x}_{i} - {o}_{j} ‖}_{2}^{2} + α {‖ H ‖}_{F}^{2} ) + λ ( {‖ XP - ( Y + H ) ‖}_{F}^{2} + γ {‖ P ‖}_{2 , 1} ) \\ s . t . \\ {\sum }_{j = 1}^{c} {h}_{ij} = 1 , 0 \leq {h}_{ij} \leq 1 \qquad ( 3 )$$

where the first term is used to learn the fuzzy membership matrix; the second term is a loss function and a term for generalization; λ is a regularization parameter, and the other parameters are the same as those described in the above expressions (1) and (2).

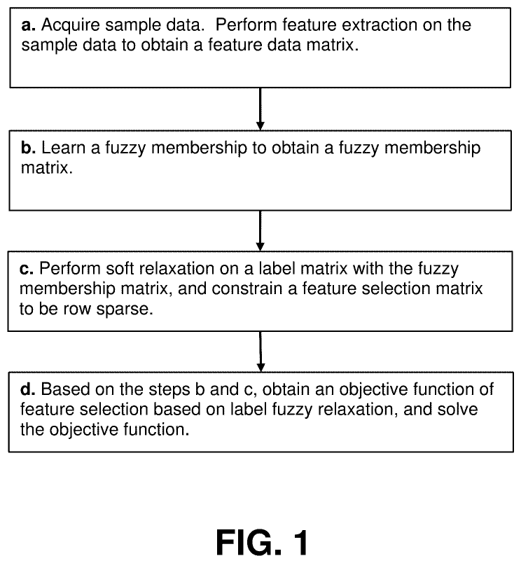

FIG. 1 shows a flowchart of the supervised feature selection method based on fuzzy label relaxation according to embodiments of the present invention. In the process of solving the expression (3), an alternative optimization method may be employed: (1) Update rule of:

$$L = {arg min}_{P} {‖ XP - ( Y + H ) ‖}_{F}^{2} + γ {‖ P ‖}_{2 , 1} \qquad ( 4 )$$

In view that the expression (4) is non-differentiable, it is transformed into the following equivalent form:

$${arg min}_{P} {‖ XP - ( Y + H ) ‖}_{F}^{2} + γ tr ( {P}^{T} Γ P ) \qquad ( 5 )$$

where

$${argmin}_{P}$$

represents a value of the independent variable p that results in a minimum value of the following expression.

$$Set F = ( Y + H ) \\ L = ⇒ tr ( {( XP - F )}^{T} ( XP - F ) ) + γ tr ( {P}^{T} ΓP ) ⇒ tr ( ( {P}^{T} {X}^{T} - {F}^{T} ) ( XP - F ) ) + γ tr ( {P}^{T} ΓP ) ⇒ tr ( {P}^{T} {X}^{T} XP ) - tr ( {P}^{T} {X}^{T} F ) - tr ( {F}^{T} XP ) + tr ( {F}^{T} F ) + γ tr ( {P}^{T} Γ P )$$

By taking a derivative of the above expression and setting the derivative to 0, the following expression is obtained:

$$\frac{\partial L}{\partial P} = 0 ⇒ {X}^{T} XP + {XX}^{T} P - {X}^{T} F - {X}^{T} F + γ ( Γ P + {Γ}^{T} P ) = 0 ⇒ 2 {X}^{T} XP - 2 {X}^{T} F + 2 γ Γ P = 0 ⇒ {X}^{T} XP + γ Γ P = {X}^{T} F ⇒ P = {( {X}^{T} X + γ Γ )}^{- 1} {X}^{T} F \qquad ( 6 )$$

(2) Update rule of oⱼ:

$$L = {arg min}_{{o}_{j}} {\sum }_{i = 1}^{n} {\sum }_{j = 1}^{c} {h}_{ij} {‖ {x}_{i} - {o}_{j} ‖}_{2}^{2} . \qquad ( 7 )$$

By taking a derivative of the above expression and setting the derivative to 0, the following deduction can be obtained, where I is an identity matrix, namely, a matrix in which all elements in the main diagonal are 1 while all other elements are 0:

$$\frac{\partial L}{\partial {o}_{j}} = 0 ⇒ {\sum }_{i = 1}^{n} ( 2 {h}_{ij} ( {x}_{i} - {o}_{j} ) ( - I ) ) = 0 ⇒ {\sum }_{i = 1}^{n} 2 ( - {h}_{ij} {x}_{i} + {h}_{ij} {o}_{j} ) = 0 ⇒ {o}_{j} = \frac{{\sum }_{i = 1}^{n} {h}_{ij} {x}_{i}}{{\sum }_{i = 1}^{n} {h}_{ij}} \qquad ( 8 )$$

(3) Update rule of H:

$$arg {min}_{H} ( {\sum }_{i = 1}^{n} {\sum }_{j = 1}^{c} {h}_{ij} {‖ {x}_{i} - {o}_{j} ‖}_{2}^{2} + α {‖ H ‖}_{F}^{2} ) + λ {‖ XP - ( Y + H ) ‖}_{F}^{2} \\ s . t . \\ {\sum }_{j = 1}^{c} {h}_{ij} = 1 , 0 \leq {h}_{ij} \leq 1 . \qquad ( 9 )$$

To simplify the expression, set dᵢⱼ=∥xᵢ−oⱼ∥₂² and R=XP−Y; then the expression can be transformed into the following form:

$$arg {min}_{H} {\sum }_{j = 1}^{c} ( {d}_{ij} {h}_{ij} + {α ( {h}_{ij} )}^{2} ) + λ {‖ R - H ‖}_{F}^{2} \\ s . t . \\ {\sum }_{j = 1}^{c} {h}_{ij} = 1 , 0 \leq {h}_{ij} \leq 1 . \qquad ( 10 )$$

Furthermore, the expression can be transformed into the following vector form (corresponding to a row vector of the matrix):

$$\begin{matrix}{arg min}_{{h}_{i}} {‖ {h}_{i} - \frac{{d}_{i}}{2 α} ‖}_{2}^{2} + λ {‖ {r}_{i} - {h}_{i} ‖}_{2}^{2} \\ s . t . {\sum }_{j = 1}^{c} {h}_{ij} = 1 , 0 \leq {h}_{ij} \leq 1.\end{matrix} \qquad ( 11 )$$

Thus, after the expression is sorted out, a Lagrange function of the expression is as follows:

$$L ( {h}_{i} , η , {θ}_{i} ) = \frac{1}{2} {‖ {h}_{i} + \frac{{d}_{i}}{2 α} ‖}_{2}^{2} + \frac{λ}{2} {‖ {r}_{i} - {h}_{i} ‖}_{2}^{2} - η ( {\sum }_{j = 1}^{c} {h}_{ij} - 1 ) - {θ}_{i}^{T} {h}_{i} , \qquad ( 12 )$$

where both η and θ are Lagrange coefficients. A solution of H can be obtained as follows according to Karush-Kuhn-Tucker conditions:

$${h}_{i} = {( \frac{λ {r}_{i} - \frac{{d}_{i}}{2 α} + η}{1 + λ} )}_{+} , \qquad ( 13 )$$

where the function ( )₊ is defined as (a)₊=max(0, a).

Although the above alternative optimization method is used in the present invention to get a solution, it can be understood that other methods in the art may also be used to get a solution.

Next, pseudo codes of the algorithm are introduced:
Inputs: X∈R^{n×d}, Y∈R^{n×c}, and regularization parameters α, γ and λ;
Outputs: a feature selection matrix P∈R^{d×c} and nSel features.
Updates are carried out below sequentially according to relevant update rules, and the following steps 1-4 are repeated:
step 1: update the fuzzy class center oⱼ;
step 2: update the membership matrix H;
step 3: update the feature selection matrix P, until (∥obj(t)−obj(t−1)∥≤10⁻⁵);
step 4: output the feature selection matrix P.
∥pᵢ∥₂, (i=1, 2, . . . , d) can be calculated according to the feature selection matrix P, and then sorting can be carried out in a descending order. The first nSel features are selected as the selected features.

In the present invention, a class structure of training samples in a high-dimensional feature space is learned with a fuzzy unsupervised learning method, the class structure is expressed by means of a fuzzy membership, label relaxation is performed by means of the fuzzy membership, and a supervised embedded feature selection framework is incorporated for solution optimization. The present invention solves the following problems in conventional methods: for example, conventional label relaxation methods only consider the criterion of “infinitely expanding the class interval”, which may lead to over-fitting easily; in contrast, in the present invention, the problem of over-fitting is solved by learning a class structure of the training samples and performing label relaxation by means of a fuzzy membership matrix that reflects the class structure. In addition, conventional label relaxation methods have poor interpretability; in contrast, the label relaxation method based on fuzzy membership in the present invention has better interpretability since it incorporates the class structure information of training samples.

The present invention has at least the following advantages over the prior art.
1) In the present invention, a class structure of training samples is learned based on fuzzy theory, and fuzzy membership is utilized to express the class structure. Such a representation method is more in line with the complex data scenarios in the real world.
2) By learning a class structure of training samples and performing label relaxation by means of a fuzzy membership matrix that reflects the class structure, the method in the present invention has better generalization performance.
3) The label relaxation method based on fuzzy membership in the present invention has better interpretability since it incorporates the class structure information of training samples.

The present invention has been proved effectively. From 2012 to 2015, 310 nasopharyngeal carcinoma patients who received radiotherapy in the Queen Elizabeth Hospital in Hong Kong, China were analyzed to ascertain whether it was necessary to reset the radiotherapy plan. For each patient, the inventor obtained 4000 features. The features were used as raw data for testing the present invention.

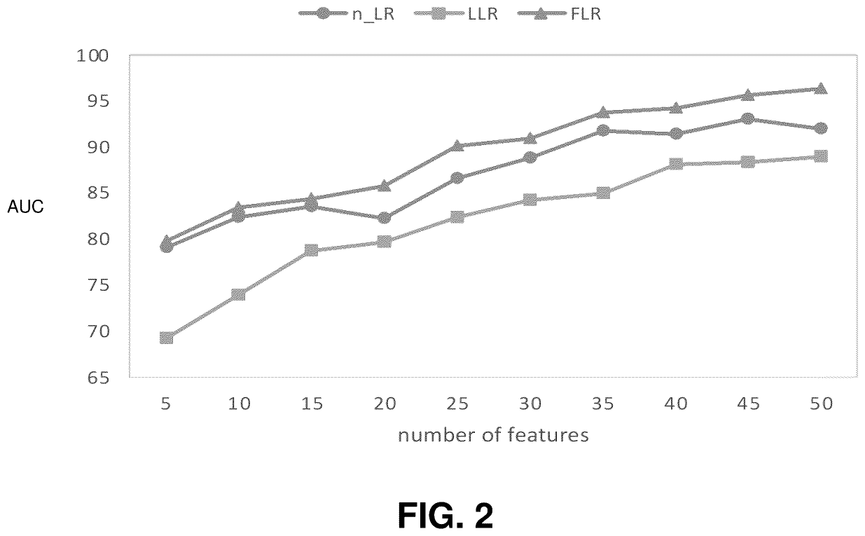

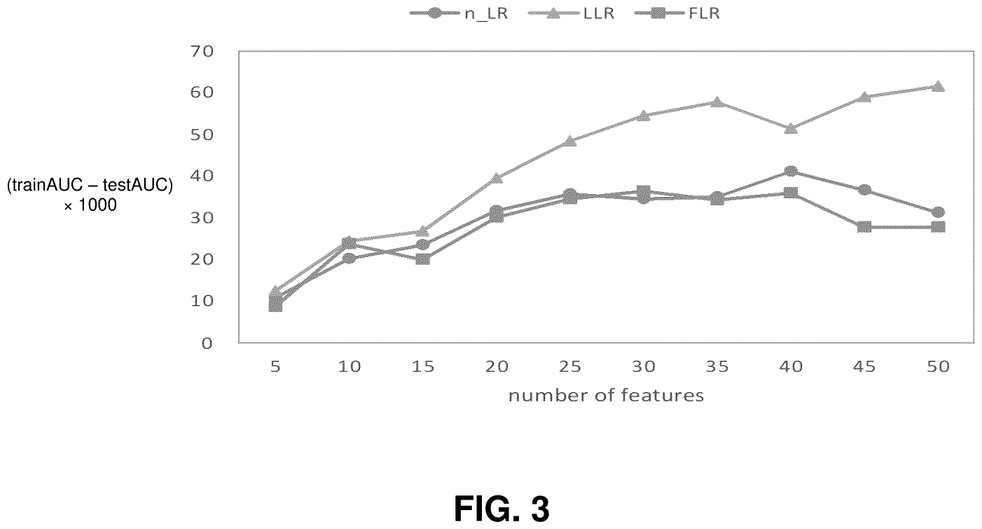

The inventors conducted experiments and obtained remarkable technical results, as shown in FIGS. 2 and 3. FIG. 2 shows the result of an ablation experiment, wherein n_LR (no label relaxation) represents the result of a device that does not use any label relaxation, while LLR (Luxury Matrix Label Relaxation) represents the result of a conventional device that uses luxury matrix label relaxation. It can be seen from FIG. 2 that the classification performance (AUC is used as the evaluation metric) of the features selected with the FLR (Fuzzy Label Relaxation) device used in the present invention is better than that of the features selected with n_LR or LLR, wherein the evaluation metric AUC is the area under curve. FIG. 3 shows the result of generalization performance. The employed metric is the absolute value of the error between training accuracy and test accuracy. The smaller the error is, the better the generalization ability is. It can be seen from FIG. 3 that the generalization performance of the FLR device in the present invention is better than that of the device that does not use any label relaxation and better than that of the conventional label relaxation device.

The present invention may also be applied to various feature selections before developing prediction or diagnosis models, such as feature selection during development of clinical diagnosis/prediction models and radiotherapy prognosis prediction models. The present invention may be applied not only to the medical field, but also to applications of feature selection in other fields such as industrial processes and image processing.

What is claimed is:

1. A feature selection method based on fuzzy label relaxation, comprising the steps of:
(a) acquiring sample data, and performing feature extraction on the sample data to obtain a feature data matrix;
(b) learning a fuzzy membership to obtain a fuzzy membership matrix;
(c) performing soft relaxation on a label matrix with the fuzzy membership matrix, and constraining a feature selection matrix to be row sparse; and
(d) based on the steps (b) and (c), obtaining an objective function of the feature selection based on fuzzy label relaxation, and solving the objective function.

2. The method according to claim 1, wherein in the step (b), the fuzzy membership is learned with the following method:
$$\begin{matrix}{min}_{{o}_{j} , H} {\sum }_{i = 1}^{n} {\sum }_{j = 1}^{c} {h}_{ij} {‖ {x}_{i} - {o}_{j} ‖}_{2}^{2} + α {‖ H ‖}_{F}^{2} \\ s . t . {\sum }_{j = 1}^{c} {h}_{ij} = 1 , 0 \leq {h}_{ij} \leq 1\end{matrix}$$
where xᵢ is the ith sample data, oⱼ is the jth class center of the sample data, and hᵢⱼ is a membership between the ith sample and the jth class center; the second term uses a square F norm to apply a regularization constraint on a membership matrix, H∈R^{n×c} is a membership matrix of the sample data features, and a is a regularization parameter of the second term in the above expression.

3. The method according to claim 2, wherein in the step (c), the objective function for the soft relaxation of the label matrix is as follows:
$${min}_{P} {‖ XP - ( Y + H ) ‖}_{F}^{2} + γ {‖ P ‖}_{2 , 1}$$
where the first term is a loss function, X∈R^{n×d} is a feature data matrix, P∈R^{d×c} is a feature selection matrix, Y∈R^{n×c} is a label matrix, and H∈R^{n×c} is the membership matrix of the sample data features; the second term applies a penalty with a L_{2,1} norm on the feature selection matrix, and γ is a regularization parameter of the second term in the above expression.

4. The method according to claim 1, wherein in the step (d), the objective function of the feature selection based on fuzzy label relaxation is as follows:
$$\begin{matrix}J = {min}_{{o}_{j} , H , P} ( {\sum }_{i = 1}^{n} {\sum }_{j = 1}^{c} {h}_{ij} {‖ {x}_{i} - {o}_{j} ‖}_{2}^{2} + α {‖ H ‖}_{F}^{2} ) + λ ( {‖ XP - ( Y + H ) ‖}_{F}^{2} + γ {‖ P ‖}_{2 , 1} \\ s . t . {\sum }_{j = 1}^{c} {h}_{ij} = 1 , 0 \leq {h}_{ij} \leq 1\end{matrix}$$
where xᵢ is the ith sample data, of is the jth class center of the sample data, hᵢⱼ is the membership between the ith sample and the jth class center, and H∈R^{n×c} is the membership matrix of the sample data features; the second term is a loss function and a term for generalization, X∈R^{n×d} is the feature data matrix, P∈R^{d×c}, is the feature selection matrix, Y∈R^{n×c} is the label matrix, and α, γ and λ are regularization parameters.

5. The method according to claim 4, wherein the objective function in the step (d) is solved with an alternative optimization method.

6. The method according to claim 5, wherein in the alternative optimization method, an update rule of P is as follows:
$$L = {arg min}_{P} {‖ XP - ( Y + H ) ‖}_{F}^{2} + γ {‖ P ‖}_{2 , 1}$$
the expression is transformed into the following equivalent form:
$${arg min}_{P} {‖ XP - ( Y + H ) ‖}_{F}^{2} + γ tr ( {P}^{T} Γ P )$$
by taking a derivative of the above expression with respect to P and setting the derivative to 0, P=(X^{T}X+γΓ)⁻¹X^{T}F is obtained, where F=(Y+H).

7. The method according to claim 5, wherein in the alternative optimization method, an update rule of oⱼ is as follows:
$$L = {arg min}_{{o}_{j}} {\sum }_{i = 1}^{n} {\sum }_{j = 1}^{c} {h}_{ij} {‖ {x}_{i} - {o}_{j} ‖}_{2}^{2}$$
by taking a derivative of the above expression with respect to oⱼ and setting the derivative to 0,
$${o}_{j} = \frac{{\sum }_{i = 1}^{n} {h}_{ij} {x}_{i}}{{\sum }_{i = 1}^{n} {h}_{ij}}$$
is obtained.

8. The method according to claim 5, wherein in the alternative optimization method, an update rule of H is as follows:
$$\begin{matrix}{arg min}_{H} ( {\sum }_{i = 1}^{n} {\sum }_{j = 1}^{c} {h}_{ij} {‖ {x}_{i} - {o}_{j} ‖}_{F}^{2} ) + λ {‖ XP - ( Y - H ) ‖}_{F}^{2} \\ s . t . {\sum }_{j = 1}^{c} {h}_{ij} = 1 , 0 \leq {h}_{ij} \leq 1\end{matrix}$$
where the expression is simplified to the following form by setting dᵢⱼ=∥xᵢ−oⱼ∥₂², and R=XP−Y:
$$\begin{matrix}{arg min}_{H} {\sum }_{j = 1}^{c} ( {d}_{ij} {h}_{ij} + {α ( {h}_{ij} )}^{2} ) + λ {‖ R - H ‖}_{F}^{2} \\ s . t . {\sum }_{j = 1}^{c} {h}_{ij} = 1 , 0 \leq {h}_{ij} \leq 1\end{matrix}$$
furthermore, the expression is transformed to the following vector form:
$$\begin{matrix}{arg min}_{{h}_{i}} {‖ {h}_{i} - \frac{{d}_{i}}{2 α} ‖}_{2}^{2} + λ {‖ {r}_{i} - {h}_{i} ‖}_{2}^{2} \\ s . t . {\sum }_{j = 1}^{c} {h}_{ij} = 1 , 0 \leq {h}_{ij} \leq 1\end{matrix}$$
after the expression is sorted, a Lagrange function of the expression is as follows:
$$L ( {h}_{i} , η , {θ}_{i} ) = \frac{1}{2} {‖ {h}_{i} + \frac{{d}_{i}}{2 α} ‖}_{2}^{2} + \frac{λ}{2} {‖ {r}_{i} - {h}_{i} ‖}_{2}^{2} - η ( {\sum }_{j = 1}^{c} {h}_{ij} - 1 ) - {θ}_{i}^{T} {h}_{i}$$
where both η and θ are Lagrange coefficients, and a solution of H can be obtained in the following way according to Karush-Kuhn-Tucker conditions:
$${h}_{i} = {( \frac{λ {r}_{i} - \frac{{d}_{i}}{2 α} + η}{1 + λ} )}_{+}$$
where the function ( )₊ is defined as (a)₊=max(0, a).

9. The method according to claim 4, wherein the objective function in the step (d) is solved with the following pseudo codes:
inputs: X∈R^{n×d}, Y∈R^{n×c}, and regularization parameters α, γ and λ;
outputs: a feature selection matrix P∈R^{d×c} and nSel features;
repeat the following steps (1)-(4):
step (1): update the fuzzy class center oⱼ;
step (2): update the membership matrix H;
step (3): update the feature selection matrix P, until (∥obj(t)−obj(t−1)∥≤10⁻⁵); and
step (4): output the feature selection matrix P;
calculate ∥pᵢ∥₂, (i=1, 2, . . . , d) according to the feature selection matrix P, and then carry out sorting in a descending order, and take the first nSel features as selected features.

10. A feature selection system based on label fuzzy relaxation, comprising:
a data set acquisition module configured to acquire sample data and perform feature extraction on the sample data to obtain a feature data matrix;
a fuzzy membership learning module configured to learn a fuzzy membership to obtain a fuzzy membership matrix;
a label matrix soft relaxation module configured to perform soft relaxation on a label matrix with the fuzzy membership matrix and constrain the feature selection matrix to be row sparse; and
an objective function solving module, which, based on the fuzzy membership learning module and the label matrix soft relaxation module, obtains an objective function of the feature selection based on fuzzy label relaxation, and solves the objective function.

11. The system according to claim 10, wherein the system utilizes the method according to claim 1.
